(19) United States
(12) Patent Application Publication
(10) Pub. No.: US 2022/0241568 A1
(43) Pub. Date: Aug. 4, 2022

(54) INTEGRATED PORTABLE APPARATUS FOR TOPICAL WOUND THERAPY, USING AMBIENT AIR FOR THE CREATION OF THREE BIOACTIVE GASES THAT INDEPENDENTLY AND SYNERGISTICALLY ASSIST IN THE RESOLUTION OF PATHOGENIC DERMATOLOGICAL CONDITIONS

(71) Applicant: Gerard V. Sunnen, New York, NY (US)

(72) Inventor: Gerard V. Sunnen, New York, NY (US)

(21) Appl. No.: 17/471,488

(22) Filed: Sep. 10, 2021

Related U.S. Application Data
(60) Provisional application No. 63/204,052, filed on Sep. 10, 2020.

(51) Int. Cl. A61M 35/00

(52) U.S. Cl. CPC A61M 35/30; A61M 2202/0216; A61M 2205/502; A61M 2202/0275; A61M 2202/0208; A61M 2205/3368

(57) ABSTRACT

A method for treating a wound includes the steps of: (1) introducing ambient air into an oxygen concentrator that increases an oxygen concentration of the ambient air and forms a first output gas; (2) introducing the first output gas into a corona discharge generator that transforms the first output gas into a second output gas by converting portions of the concentrated oxygen into reactive oxygen species (ROS), and converting portions of the nitrogen gas into nitrogen reactive species (NRS); and (3) delivering the second output gas into a multi-gas compartment within a treatment device in which the wound is positioned.

### CROSS REFERENCE TO RELATED APPLICATION

The present application claims priority to and the benefit of U.S. patent application Ser. No. 63/204,052, filed Sep. 10, 2020, which is hereby incorporated by reference in its entirety.

### BACKGROUND

The use of ozone/oxygen gaseous mixtures has been described in reference to the treatment of external pathogenic conditions (Sunnen, U.S. Ser. No. 09/126,504, which is hereby incorporated by reference in its entirety). In 2000, the present inventor was granted U.S. Pat. No. 6,073,627, “Apparatus for the application of ozone/oxygen for the treatment of external pathogenic conditions.” which is hereby incorporated by reference in its entirety. Also, an application was submitted, “Apparatus and Method for Precise Ozone/Oxygen Delivery Applied to the Treatment of Dermatological Conditions, Including Gas Gangrene and related Disorders.” U.S. Ser. No. 11/110,066, which is hereby incorporated by reference in its entirety.

Despite enormous advances in medicine relative to the treatment of many dermatological conditions, far too many wounds remain frustratingly recalcitrant to healing and resolution. There are several reasons for this state of affairs. Demographically, the aging population is confronted with an increasing prevalence of chronic diseases, such as diabetes and circulatory disorders. These conditions all too often lead to tissue breakdown, skin pathologies and chronic infections. Amputations sometimes follow, with enormous implications for functionality and mental health consequences.

Other factors implicate the microorganism ecology of contemporary wounds. Indeed, microbes do not stand still. They deftly evolve, sometimes rapidly, showing new resistance and novel adaptation. A medication that was effective one year may not be the next. Indeed, chronically infected wounds often harbor numerous types of microorganisms—bacteria, fungi, viruses and parasites—belonging to disparate microbial families that often need multiple therapeutic agents and modalities, challenging clinical care.

There is therefore a need for an improved wound treatment strategy and related equipment that addresses and overcomes the deficiencies noted above.

### SUMMARY

The present disclosure is pertinent to current needs in that it proposes wound treatment strategies that make use of both the antimicrobial, and the tissue health-enhancing properties of three selected gases normally found in nature, all intrinsic to normal physiology. Oxygen is essential to normal metabolism. Ozone is produced by the body's macrophages and neutrophils to kill microorganisms. And nitric oxide acts as a vasodilator and a cell-to-cell signaling molecule. Via their oxidative potential, oxygen (O2) and oxygen reactive species (ROS) disrupt the life cycles of microbes that commonly colonize wounds. Concomitantly, nitrogen reactive species (NRS), including nitric oxide, promote tissue circulation and activate immune functions.

The mixture of these gases disrupts microbial viability not only via direct action, but also by countering common microbial defenses such as bacterial biofilms and tissue-destroying bacterial toxins. These gases, because they hold such prominent roles in normal physiology, have also demonstrated capacities to assist healthy tissue integrity through such mechanisms as the stimulation of microcirculation and the normalization of cellular function.

The present disclosure proposes an integrated self-contained unit that derives its final product, namely a controlled mixture of externally applied therapeutic gases, from ambient air. Via selective alteration of normal air's composition via the application of electrical energy, the gas components are modified so as to assume appropriate concentrations for wound healing.

The therapeutic gases are applied externally. Wounds under treatment are encased within a treatment envelope made of resistant plastics such as those derived from silicones or polyethylenes. These materials resist the oxidizing actions of ROS and NRS and serve to create a gaseous environment for the wound care.

The present disclosure presents an innovative step for the following reasons: whereas in previous systems the gas source was pure oxygen—usually provided by a medical or industrial grade oxygen cylinder—this system proposes the deliberate inclusion of small amounts of nitrogen for producing molecules in the nitrogen family capable of assisting in wound healing. Using ambient air as a gas feed not only makes the system portable and versatile, but also allows access to one of air's major component: Nitrogen.

In one embodiment, a method for treating a wound includes the steps of: (1) introducing ambient air into an oxygen concentrator that increases an oxygen concentration of the ambient air and forms a first output gas; (2) introducing the first output gas into a corona discharge generator that transforms the first output gas into a second output gas by converting portions of the concentrated oxygen into reactive oxygen species (ROS), and converting portions of the nitrogen gas into nitrogen reactive species (NRS); and (3) delivering the second output gas into a multi-gas compartment within a treatment device in which the wound is positioned.

### BRIEF DESCRIPTION OF THE DRAWING FIGURES

FIG. 1 is a schematic of a system in accordance with one embodiment of the present disclosure.

### DETAILED DESCRIPTION OF CERTAIN EMBODIMENTS

It has been amply demonstrated that, in the armamentarium of the body's defenses, both reactive oxygen species (ROS) and nitrogen reactive species (NRS) assume important functions relative to antimicrobial action, and general physiology including circulation and immune system performance.

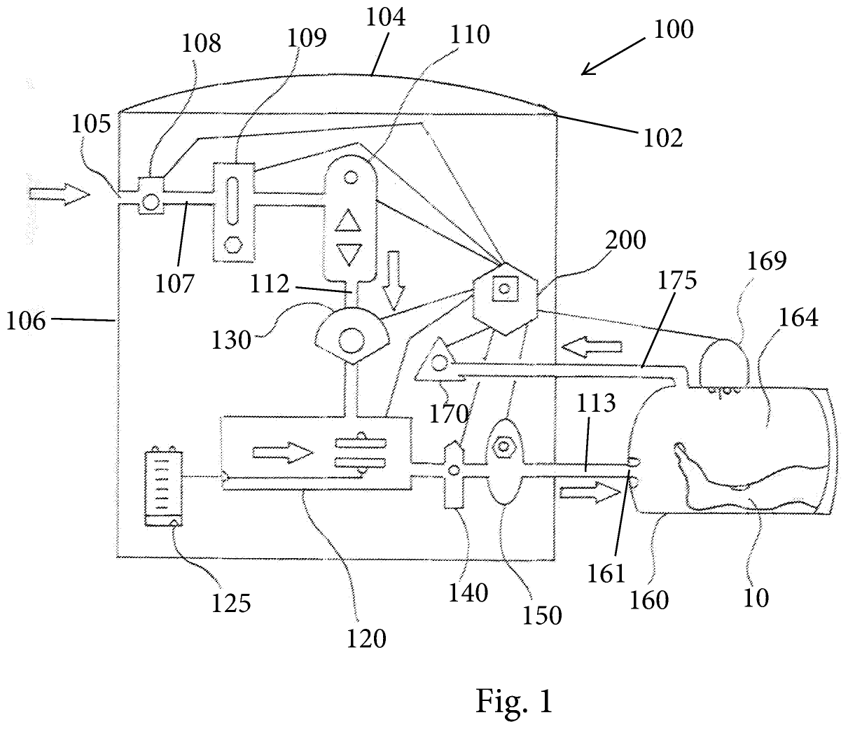

FIG. 1 illustrates a system 100 that is configured to process ambient air to yield the ROS and NRS and produce a controlled gaseous mixture that is then introduced into a gas compartment that surrounds the wound to provide the advantages described herein.

Processing ambient air to yield ROS and NRS One main advantage of the system 100 is it ability to process ambient air. In particular, the system 100 and related method are configured such that ambient air's constituents are processed so that both ROS and NRS are generated in proportions optimal for wound resolution as described herein. While the present disclosure uses the term “system,” it will be appreciated that the arrangement of parts disclosed herein can equally be thought of as defining an apparatus or device that controllably generates a gaseous environment comprising both ROS and NRS for wound treatment.

In past practices, topical healing gases were generated by feeding oxygen to a high-energy field supplied by a corona discharge generator. In the system herewith presented. ambient air is used to create desired gaseous mixes, thus obviating the use of gas cylinders. This approach increases this technology's availability for patient populations in need, including those in nursing homes, rural health facilities and even remote military clinics.

One main difference and advantage of the present system relates to the addition of the therapeutic potential of nitrogen reactive species (NRS) in wound healing, including nitric oxide (NO), a recognized cell messenger, neurotransmitter, anti-oxidant and vasodilator. Importantly for the purpose of this invention, NRS, in proper concentrations, also possesses notable antimicrobial action.

The present system has the capacity to modulate the proportion and concentration of these gases by integrating sensors for oxygen, ozone and nitric oxide in the final gaseous product. Servomechanisms can be programmed to enable treatment adaption to the individual presentation of wounds and to their evolution.

As described in more detail below, the present system preferably incorporates a humidity sensor, a dehumidifier and a humidity generator. The rationale for this feature is that the addition of water vapor to the gaseous mix may, in certain clinical situations, bolster therapeutic advantage. On the other hand, too much humidity in the context of creating nitrogen species can generate compounds, such as acids that, in higher concentrations, may be toxic. With dehumidification, water vapor may be withdrawn during the corona discharge process. Humidity, on the contrary, may be added to the final product. In this scenario, water vapor, mixed with nitrogen reactive species, creates weak nitric acid vapor that, in appropriate concentrations, can assist in the removal of bacterial biofilms.

FIG. 1 illustrates one exemplary system 100. The system 100 includes a main housing 102 that encloses and contains the working components of the system 100. The main housing 102 can have any number of different shapes and sizes. In the illustrated embodiment, the main housing 102 has a flat bottom to permit it to rest on a support surface, such as the floor or a table, etc. Alternatively, the housing 102 can even be mounted to a wall. The housing 102 has a top 104 and a side 106.

Along the housing 102 (e.g., along the side 106), the system 100 includes an ambient air intake 105, such as an air intake connector or port, that permit attachment of a conduit or the like through which ambient air can be delivered to the working components of the system 100. It will be appreciated that a pump or the like can be used to pump the ambient air into the system 100.

For sake of illustration, a first conduit 107 is shown within the housing 102 that is in fluid communication with the air intake 105. Along the first conduit 107, there is preferably an air filter 108 for filtering the incoming ambient air. There can also be a fan and flow meter 109 that is located along the first conduit 107. The fan and flow meter 109 regulates the inflow and sends ambient air downstream to other equipment as described below.

Processing Ambient Air to Yield ROS and NRS

In the process of transforming ambient air to a wound-healing gas mixture, the present system 100 first enhances oxygen concentration using an oxygen concentrator 110 that is contained in the housing 102.

Oxygen Concentrator 110

The oxygen concentrator 110 is configured to concentrate the oxygen concentration of the air that is introduced into the oxygen concentrator 110. The oxygen concentrator 110 operates by withdrawing nitrogen from the ambient air via the oxygen concentrator 110 component of the present system 100.

As is known, the oxygen concentrator 110 operates by receiving air, purifying air, and the distributing the newly formed air. Generally, before it goes into the oxygen concentrator 110, the ambient air is made up of 80 percent nitrogen and 20 percent oxygen. The oxygen concentrator 110 uses that ambient air then it comes out as 90 to 95 percent pure oxygen and 5 to 10 percent nitrogen for general use. The five steps of the oxygen concentrator process are as follows: 1. takes air from the surrounding environment (room); 2. compresses the oxygen; 3. takes out nitrogen from the air; 4. adjusts the way the air is delivered; and 5. delivers the purified air.

In one exemplary embodiment of the present process and with a high efficiency for nitrogen removal, ambient air may be converted using the oxygen concentrator 110 to a mixture that may reach upward of 96% oxygen concentration (e.g., between 93-96% oxygen). Once processed by the system's oxygen concentrator 110, its outflow yields mostly oxygen, the rest nitrogen and minuscule amounts of carbon dioxide.

Based on the foregoing, it will be appreciated that the source of gas for the oxygen concentrator 110 is the ambient air. As mentioned, the oxygen concentrator 110 essentially removes nitrogen from air, reducing it from its normal concentration of approximately 80%, to 4% in efficient units. Oxygen outflow from the unit can thus attain 96% and above.

The oxygen concentrator's properties depend on the ability of microporous aluminosilicate minerals (Zeolite) to bind nitrogen. Oxygen concentrator's efficiency works on the ingenious interplay of two Zeolite-filled sieves working in toggle fashion so that as one sieve becomes saturated with nitrogen, the other takes over. Once saturated, sieves are flushed out with oxygen and resume their tandem relay functions.

The gas flow output of the oxygen concentrator varies according to its intended usage. For purposes of this invention, however, an acceptable gas flow ranges from 0.5 liter per minute to 5 liters per minute.

Corona Discharge Generator 120

There are several ways to impart energy to gases. The method chosen for this system incorporates corona electrical discharge because this solid-state technology has proven to be efficient, reliable and durable. An alternate power source could use ultraviolet technology for the system. An energy source 125, in this case a rechargeable battery or a connection to main lines provide the power to the system and more particularly power the corona discharge generator 120. A high voltage transformer brings the incoming current to proper higher tension, which is then distributed to apposed electrodes. The corona generator 120 is essentially a high voltage energy field through which the outflow gas mixture from the oxygen concentrator 110 passes through.

The corona energy alters the gas outflow components coming from the oxygen concentrator 110. Since this outflow contains mostly oxygen, some proportion of nitrogen and minute doses of carbon dioxide, the electrical activation of this outflow gas by the generator 120 yields three main gases, each of which have bioactive properties. Principally, these comprise the oxygen (O2) that remains unchanged by the process, ozone (O3), and nitric oxide (NO). There are, however, small yet bioactive amounts of derivative products, classified as members of the ROS and NRS families. Many of these products exist transiently yet possess documented antimicrobial capacity and incompletely clarified biological effects, all legitimate queries for research.

The corona discharge generator 120 can produce ozone up to 5% per volume, and NO in air (0 to 100 ppm [0 to 20 micrograms/g of air]).

Some corona discharge derivatives have little or no effect on wound dynamics and can be regulated by mitigation technologies, as is the case with nitrogen dioxide.

The corona generator 120 is specifically designed for use in wound resolution therapy. As such, its capabilities for precise adjustments of gas flow, amperage and voltage regulation, humidity and temperature compliance respect the medical needs for precision. This makes for enhanced capacity to deliver desired proportions of main gases as well as ROS and NRS to the treatment envelope encasing the wound as described in more detail below.

In the illustrated embodiment, the concentrated, purified air that exits the oxygen concentrator 110 flows into a second conduit 112. This gaseous mixture flows through the second conduit 112 and is then fed to a corona discharge generator 120 that imparts it with electrical energy. As is known, a corona discharge generator 120 can be configured to create the ROS (e.g., ozone (O3)) from oxygen (O2). Any electrical discharge, or spark will create ozone. The spark will split the oxygen molecule (O2) found in ambient air into elemental oxygen (O). These Oxygen atoms will quickly bind to another oxygen molecule (O2) to form ozone (O3). The electrical energy used in ozone generation splits the oxygen molecule. In a corona discharge ozone generator, the electrical discharge will take place in an air gap within the corona cell designed specifically to split the oxygen molecule and produce ozone. In this air gap a dielectric is used to distribute the electron flow evenly across this gap to spread the electron flow to as great a volume of oxygen as possible.

The corona discharge generator 120 thus has an inlet that receives the concentrated, purified air from the oxygen concentrator 110 and has an outlet for discharging the newly formed gas mixture.

It is during this phase that portions of the oxygen are converted to reactive oxygen species (ROS), and that portions of the nitrogen gas are converted to nitrogen reactive species (NRS). The gas outflow from the system will contain proportions of different gases, clinically chosen via the adjustments made to the oxygen concentrator 110 and to the corona discharge generator 120. As described herein, the user can thus control the manner in which one or both of the oxygen concentrator 110 and the corona discharge generator 120 operate.

The system's gas outflow from the corona discharge generator 120 thus contains a mixture composed of oxygen with reactive oxygen species (ROS)—chemically reactive oxygen-containing molecules. Normally produced by normal metabolism, they are ozone, singlet oxygen, hydroxyl radical, peroxyl radical, peroxides and superoxide. Also included are nitrogen (N) and nitrogen reactive species (NRS) (gases), namely: nitric oxide, nitrogen dioxide and higher oxides of nitrogen, as well as minute proportions of carbon dioxide.

The configuration of this gas mixture is adjusted by methods that proportion the various gas component concentrations to the treatment of the specific medical condition. A wound whose bacteriological analysis shows high potential for toxin formation may, for example, be initially administered higher concentrations of topical ozone to oxidize the said toxin (i.e., a higher ratio of ROS/NRS). A wound presenting with a bacterial biofilm may be prescribed higher proportion of NRS (i.e., a higher ratio of NRS/ROS) to disrupt fibrin in the biofilm.

Oxygen, ozone, and nitric oxide have known physiological effects that encourage wound resolution via a variety of mechanisms, mainly microbial inactivation and enhanced vascular perfusion. The unit delivers this gaseous mixture to a fitted envelope encasing the wound under treatment. Wound exposure time versus gas concentration application is predicated on serial clinical evaluation protocol.

The present system 100 proposes a method of treating wounds with variable mixture of oxygen, ozone, and nitric oxide, adjustable in proportions predicated on the pathology under treatment. The proprietary mixture of these gases, topically applied to skin wounds and calibrated according to their clinical manifestations, embodies advantages that are not achieved by any of the individual gases used alone. Oxygen, by itself, is approved for wound healing, especially if it is administered under pressure, as in hyperbaric therapies. Adding small amounts of ozone to the oxygen, an entirely safe option, allows for added antimicrobial action and the addition of nitric oxide promotes immune cellular activation and vasodilation for increased wound perfusion.

The present system 100 and method may be directed to the treatment of acute wound conditions for the prevention of infection. It may be applied for the treatment of chronic skin conditions such as diabetic ulcers, vascular ulcers, decubitus skin ulcers and burns. Due to its self-containment and portability, the present system 100 is ideally suited for use to treat patients who are homebound, in nursing homes and in chronic care and rehabilitation facilities.

The system's self-containment makes it suitable for use in acute care situations where different components may be cumbersome to assemble. Such situations may include war zones and extreme rural and isolated environments. Because access to electrical supplies can be limited, options for solar panel feeds are incorporated in the apparatus.

The system 100 does not require the presence of an external oxygen supply, as in a tank, because ambient air and an oxygen concentrator 110 feed the working components of the system 100. In fact, this feature is an essential embodiment of the invention because the oxygen concentrator's output is such that nitrogen remains as a (small) component of its gaseous outflow.

The gas outflow from the oxygen concentrator 110 contains adjustable proportions of oxygen and nitrogen using traditional equipment such as valves and a user interface. This mixture is then fed to the ozone generator (corona discharge generator) 120. In this case, however, it is more accurately referred to as an ozone/nitric oxide (O3/NO) generator 120 since one of the roles of the generator 120 is to generate these species (ozone and nitric oxide gas for wound treatment). This generator 120 imparts variable electrical energy to the gas mixture to produce desired proportions of three different gases that each have wound resolution properties, but in conjunction, have synergetic effects on microorganism inactivation on one hand, and energized wound physiology on the other.

The system 100 also incorporates an oxygen analyzer, an ozone analyzer, and a nitric oxide analyzer. Thus, an accurate gauging of oxygen, ozone and nitric oxide gas concentrations can be ascertained, as they are applied to wounds in real time. As described herein, these analyzers can be in the form of one or more sensors that monitor the relative concentrations of these components within the gas compartment.

Humidification/Dehumidification

The gas circuit of the present system 100, from air intake to delivery into the wound-encasing treatment space (e.g., an envelope described herein), requires humidity control. As shown in FIG. 1, before entering the corona discharge generator 120, incoming water vapor may be regulated by sensors that communicate to a first humidifier/dehumidifier unit 130 that is configured to act on the incoming air and either add or remove humidity therefrom. The first humidifier/dehumidifier unit 130 is thus located within the second conduit 112 as shown. Humidity may thus be adjusted before entry into the corona discharge generator 120, as well as after the exit from the corona discharge generator 120. More specifically, a second humidifier/dehumidifier unit 140 can be provided in a third conduit 113 that is in fluid communication with the outlet of the corona discharge generator 120 and receive the gas outflow from the corona discharge generator 120. Like the first humidifier/dehumidifier unit 130, the, a second humidifier/dehumidifier unit 140 is configured to act on the incoming air and either add or remove humidity from the gas outflow from the corona discharge generator 120.

The first humidifier/dehumidifier unit 130 is thus for regulation of water vapor content before it arrives at the corona discharge generator 120. Oxygen Reactive Species (ORS) and to Nitrogen Reactive Species (NRS). Before the outflow gas (Oxygen Reactive Species (ORS) and to Nitrogen Reactive Species (NRS)) gets to the wound under treatment, humidity levels are once more adjusted by the second humidifier/dehumidifier unit 140.

Temperature Controller or Regulator 150

The system 100 can further include the temperature controller or regulator 150 that is downstream of the corona discharge generator 120 and located within the third conduit 113. Any number of different types of temperature controllers, such as heaters and cooling units that chill adjacent conduits, can be used to either heat or cool the outgoing gas mixture to reach optimal temperature levels for wound healing. In one embodiment, an electric heater can be used as well as an electric cooler that can be provided in a common device. In other words, the temperature controller 150 can be in the form of a heat exchanger that relies of flowing fluid to either cool or hear the surrounding environment. Since the gaseous mixture is exposed to the wound, the temperature of the gaseous mixture should be regulated to provide a comfortable temperature and/or be at a temperature (e.g., an elevated temperature relative to ambient air temperature) that promotes wound healing.

Treatment Device 160

The treatment device 160 can be broadly thought of as being a controlled space in which the wound is positioned for treatment with the outflow gas that is generated by the system 100. The treatment device 160 is defined by a housing or outer structure 162 that defines a multi-gas compartment 164 in which the wound is placed. The treatment device 160 can thus take many different forms and sizes and shapes. In the illustrated embodiment, the treatment device 160 is in the form of a treatment envelope where the wound is exposed to the multi-gas compartment 164.

As shown, the treatment device 160 has an inlet 161 that is in fluid communication with the third conduit 113 and is in fluid communication with the multi-gas compartment 164.

As previously mentioned, the treatment space (i.e., the multi-gas compartment 164) contains sensors and the like for monitoring the treatment space. These sensors, generally shown at 169, are configured to convey intra-envelope gas concentration readings to a main controller 200 that is discussed below. As shown, the sensors 169 can be a plurality of discrete sensors that are located internally along the multi-gas compartment 164. For example, the plurality of sensors 169 can be located along one or more walls (e.g., a top wall) of the treatment device 160. Each of the sensors 160 is in direct communication with the main controller 200 using conventional techniques, such as a wired connection or wireless connection.

In one embodiment, the plurality of sensors 169 can include gas sensors, such as an oxygen sensor, an ozone sensor, and a nitric oxide to measure these gases in the final gaseous mixture that is delivered into the multi-gas compartment 164.

As also described herein, while the wound 10 under care shown in FIG. 1 is located along a leg, it will be appreciated that this is merely exemplary and the wound 10 can be located in other locations along the body.

Main Controller/Microprocessor Unit 200

The main controller/microprocessor unit 200 receives data from measurements and data from the other working components of the system 100, such as the plurality of sensors 169. As is known, the main controller/microprocessor 200 communicates with the other working components, such as the sensors 169, using conventional techniques, such as conventional communication protocol and can be wired or wireless connections. Computer programs (and other executable instructions) and data can be stored on a machine-readable medium that is accessible by one or more processors for providing functionality shown and described herein. Various forms of computing devices are accessible to a network and can communicate over the network to the various machines that are configured to send and receive content, data, as well as instructions that, when executed, enable receipt of the measurements from the sensors 169 and also permits control signals to be sent to the heater/cooler unit and/or the humidifier/dehumidifier, etc. The lines shown in FIG. 1 between the main controller 200 and the individual working components indicate communication, either wired or wireless, between these parts of the system 100.

The content and data can include information in a variety of forms and can include embedded information such as links to other resources on the network, metadata, and/or machine executable instructions. Each computing device can be of conventional construction, and while discussion is made in regard to servers that provide different content and services to other devices, such as mobile computing devices, one or more of the server computing devices can comprise the same machine or can be spread across several machines in large scale implementations, as understood by persons having ordinary skill in the art. In relevant part, each computer server has one or more processors, a computer-readable memory that stores code that configures the processor to perform at least one function, and a communication port for connecting to the network. The code can comprise one or more programs, libraries, functions or routines which, for purposes of this specification, can be described in terms of a plurality of modules, residing in a representative code/instructions storage, that implement different parts of the process described herein. Further, computer programs (also referred to herein, generally, as computer control logic or computer readable program code) can be stored in a main and/or secondary memory and implemented by one or more processors (controllers, or the like) to cause the one or more processors to perform the functions of the invention as described herein. In this document, the terms “memory,” “machine readable medium,” “computer program medium” and “computer usable medium” are used to generally refer to media such as a random access memory (RAM); a read only memory (ROM); a removable storage unit (e.g., a magnetic or optical disc, flash memory device, or the like); a hard disk; or the like.

Alternatively, gas sensors 169 may be placed directly at the outflow gas exit of the O3/NO generator.

Ozone Destructor Device 170

An outlet conduit 175 is in fluid communication with the multi-gas compartment 164. The outlet conduit 175 leads to an ozone destructor device 170. The ozone destructor 170 converts ozone to pure oxygen, releasing it to ambient air. Gas outflow from the treatment device (envelope) 170 needs to be sanitized before release into the ambient air. The system 100 deactivates ROS present in the off-gas via the incorporation of an ozone destructor. Manganese dioxide (MNO2) canisters or thermo-catalytic destruct units accelerate ozone's natural regression to pure oxygen. Indeed, the half-life of ozone at room temperature is approximately one hour, but with destructors, ozone life span can be greatly shortened. Any number of traditional ozone destructor devices 170 can be used. As shown, the ozone destructor device 170 can contain a sensor that is in communication with the master controller 200 so that the level of ozone and/or the level of oxygen gas (i.e., the amount of ozone and/or the amount of oxygen gas) is monitored continuously to make sure the ozone destructor device 170 is operating properly.

Ozone and ROS as Anti-Microbial Agents in Wound Care

Infected wounds, and especially chronic lesions, may show a wide spectrum of pathogen growth, including bacteria. viruses, fungi, and protozoa.

The anti-pathogenic effects of ozone have been substantiated for several decades. Its pan-pathogen properties are universally recognized. Coliforms such as Salmonella, show marked sensitivity to ozone inactivation. Other bacterial organisms susceptible to ozone's disinfecting properties include Streptococci, Staphylococci, Shigella, Legionella, Pseudomonas, Yersinia, Campylobacter, Mycobacteria, Klebsiella, and Escherichia coli.

Ozone destroys both aerobic, and importantly, anaerobic bacteria, which are mostly responsible for the devastating sequelae of complicated infections, as exemplified by decubitus ulcers and gangrene.

The mechanisms of ozone bacterial destruction need to be further elucidated. It is known that the cell envelopes of bacteria are made of polysaccharides and proteins, and that in Gram-negative organisms, fatty acid alkyl chains and helical lipoproteins are present. In acid-fast bacteria, such as Mycobacterium tuberculosis, one third to one half of the capsule is formed of complex lipids (esterified mycolic acid, in addition to normal fatty acids), and glycolipids (sulfolipids, lipopolysaccharides, mycosides, trehalose mycolates).

The high lipid content of the cell walls of these ubiquitous bacteria may explain their sensitivity, and eventual demise, in the face of ozone exposure. Ozone may also penetrate the cellular envelope, directly affecting cytoplasmic integrity and genetic capacity.

Viruses' Susceptibility to Reactive Oxygen Species

Numerous families of viruses including Coronaviruses, Poliovirus 1 and 2, Influenza, HIV, Herpes, rotaviruses, Norwalk virus, Parvoviruses, and Hepatitis B and C, among many others, are susceptible to the virucidal actions of ozone. Ozone's virucidal effects have centered upon its propensity to splice lipid molecules at sites of viral multiple bond configuration. Indeed, once the lipid envelope of the virus is fragmented, its DNA or RNA core cannot survive.

Non-enveloped viruses (Adenoviridae. Picornaviridae (poliovirus), Coxsachie, Echovirus, Rhinovirus, Hepatitis A, D, and E, and Reoviridae (Rotavirus), have also be studied in relation to ozone inactivation. Viruses that do not have an envelope are called “naked viruses.” They are constituted of a nucleic acid core (made of DNA or RNA) and a nucleic acid coat, or capsid, made of protein. Ozone, however, aside from its well-recognized action upon unsaturated lipids, can interact with certain viral proteins and amino acids. When ozone comes in contact with capsid proteins, protein hydroxides and protein hydroperoxides are formed.

Viruses have no protection against oxidative stress. Normal mammalian cells, on the other hand, possess complex systems of enzymes (e.g., superoxide dismutase, catalase, peroxidase) that tend to ward off the nefarious effects of free radical species from oxidative challenges. It may thus be possible to treat infected tissues with ozone while respecting the integrity of their healthy cell components.

Fungal Susceptibility to ROS

Fungi families are frequent invaders of wounds. Inhibited and destroyed by exposure to ozone are Candida, Aspergillus. Histoplasma, Actinomycoses, and Cryptococcus. The cell walls of fungi are multilayered and are composed of approximately 80% carbohydrates and 10% of proteins and glycoproteins. The presence of many disulfide bonds has been noted, making this a possible site for oxidative inactivation by ozone.

Protozoa Sensitivity to ROS

Protozoan organisms often colonize wounds. Ozone inactivates Giardia, Oyptospohdium, and free-living amoebas, namely Acanthamoeba, Hartmonella, and Negleria. The exact mechanism through which ozone exerts anti-protozoan action has yet to be elucidated.

Cutaneous Effects of Pure Oxygen Mixtures

The positive effects of oxygenation on many dermatological conditions have long been established, and form the basis for the use of hyperbaric oxygen treatment. Oxygen diffuses into the tissues, raising their oxidation-reduction potential thus directly inhibiting the growth of anaerobic bacteria.

While oxygen itself inhibits microorganism growth, ozone, as an enhanced acceptor of electrons, is much more potent in its anti-microbial action. While the most likely beneficial effect of external ozone administration is pathogen inactivation. it is important to note ozone's contribution to healing through its physiological actions on normal tissues. Ozone dilates the arterioles in wounds, thus stimulating the inflow of nutrients, immune cells and molecules. By similar mechanisms, ozone accelerates the outflow of waste products, including toxins. Ozone oxidizes bacterial toxins and disrupts bacterial defenses such as biofilms.

External Medical Conditions Benefited by Topical Oxygen/Ozone Gas Mixtures

In view of the above-mentioned principles of ozone/oxygen's biological properties, and nitric oxide's tissue the present invention seeks to harness this therapeutic potential, not only for the treatment of several dermatological conditions, but also for their prevention. The following is a list of pathologic dermal conditions that may be addressed by external ozone/oxygen therapy. The most serious is gangrene, and the most ominous is gas gangrene.

Gangrene and Gas Gangrene

Gas gangrene may be a rapidly fatal complication of traumatic injuries such as automobile accidents and war injuries, surgical incisions and wounds, burns, and decubitus ulcers, among many other conditions. Predisposing factors include diabetes, arteriosclerosis, surgeries involving the intestinal tract, and septic abortions.

Gas gangrene, also known as necrotizing fasciitis, myositis, and myonecrosis is feared because of the rapidity of its evolution and the galloping and irreversible demise of affected tissues.

Several bacterial species are implicated in this process, the most common belonging to Clostridium families. These anaerobic bacteria thrive in the absence of oxygen, feeding on glycogen and sugars, producing lactic acid and gases such as methane, carbon dioxide, and hydrogen, among others. They also produce toxins capable of causing hemolysis, renal failure, and shock.

Other bacterial species are implicated in gas gangrene aside from Clostridium, including Enterobacteria, E. coli, Proteus, Group A streptococcus, Staphylococcus, Vibrio, Bacteroides, and Fusiforms. Ozone is effective in inactivating all these anaerobic and aerobic families.

Poorly Healing Infected Wounds

This category of wound has, by definition, not yet reached the status of chronicity due to a combination of circulatory compromise and infective onslaught. In fact, this category of wound may simply be post-surgical, and only potentially prone to infection.

The use of topical ozone therapy in these cases may be solely preventive, aimed at improving circulation on one hand, and inhibiting the proliferation of potentially infective organisms on the other.

Wounds that heal in an indolent manner are frustratingly difficult to master. Generally speaking, poorly healing wounds owe their definition to their chronicity, which is most commonly caused by the profusion and variety of offending organisms they harbor.

War/Com Bat Wounds

War wounds often present complex treatment challenges. Healing is often complicated by the presence of shrapnel and other foreign bodies and bone debris. Infection is favored by hot weather and high humidity.

Ozone/oxygen external application offer excellent prophylaxis for infectious processes made likely by the special nature of war wounds.

Decubitus Ulcers

Decubitus ulcers often arise when patients remain in restricted positions for prolonged periods of time, as in beds and wheelchairs. The pressure exerted upon skin contact points compresses the dermal arterioles preventing the proper perfusion of tissues. This leads to tissue oxygen starvation, impaired skin resilience, and to the eventual breakdown of the skin integrity. An expanding ulcer develops, usually infected by a spectrum of pathogenic organisms. At times the breakdown is so severe that the ulcer reaches the bone, ushering osteomyelitis.

The treatment of decubitus ulcers requires a multidisciplinary approach, including surgical, pharmacological, and physiological interventions. Topical antibiotics, often failing to penetrate the depth of the wound, are active only against a limited spectrum of organisms, induce resistance, and not infrequently cause secondary dermatitis in their own right.

Circulatory Disorders

This class of disorders has one common denominator, namely the impaired circulation to tissues via compromise of vascular integrity. A prototypic disease is diabetes. Diabetes manifests both vascular disturbances to many organ systems (e.g. retina, kidney), and disruptions to carbohydrate metabolism. In cases where diabetes affects the peripheral circulation, tissues such as the dermis become vascularly compromised, and thus more prone to injuries and infections.

Diabetic ulcers frequently develop following abrasions, contusions, and pressure injuries. These ulcers, not unlike decubitus ulcers, are notoriously difficult to treat. Topical ointments can only address a minor spectrum of putative infectious organisms. These same organisms, furthermore, may rapidly develop antibiotic resistance.

Serially applied oxygen/ozone/nitric oxide topical therapy inactivates most, if not all, offending pathogens and these same pathogens are unable to build a resistance to its effects.

Arteriosclerosis is a condition marked by the thickening and hardening of the vascular tree. The normal pliability and patency of blood vessels is compromised, leading to impaired circulation in many organ systems. In the face of reduced peripheral circulation (e.g., arteriosclerosis obliterans), skin disorders may include trophic changes (e.g., dry hair, shiny skin) apt to injury and eventual ulcer formation.

Lymphatic Diseases

The lymphatic system regulates fluid equilibration within the body and, most importantly, offers infection defense.

Lymphedema is a condition caused by blockage to lymphatic drainage. It may be secondary to trauma, surgical procedures, and infections (e.g., streptococcal cellulitis, filariasis, lymphogranuloma venereum).

Increasingly common is lymphedema resulting from surgical removal of lymph nodes following surgery for breast cancer. The affected arm in these patients is likely to be chronically swollen and indurated. Most alarming, however, is the occurrence of infections following even minor injuries to the arm. Injuries are then much more likely to become infected due to the absence of lymphatic system defenses. In these cases, intensive topical wound care is initiated, and systemic antibiotic treatment is prescribed.

Topical oxygen/ozone/nitric oxide treatment applied in a timely fashion to the affected hand or arm may prevent secondary infection; and it may avoid the use of systemic antibiotics.

Fungal Skin Infections

Fungi are present on human skin in a quasi-symbiotic relationship. Candida, Aspergillus, and Histoplasma, for example, are often found on intact skin, without causing clinical problems. However, under certain conditions, the normal balance of the dermis is disturbed, allowing superficial fungi to proliferate. Tinea capitis is manifested by pustular eruptions of the scalp, with scaling and bald patches. Tinea cruris is a fungal pruritic dermatitis in the inguinal region

Serial topical oxygen/ozone/nitric oxide applications have already shown marked success in eradicating the most chronic and stubborn fungal skin conditions. Tri-gas therapy may enhance this effectiveness, and requires research.

Burns

Thermal burns are divided into first, second, and third degrees, depending upon the depth of tissue damage. First-degree burns are superficial, and include erythema, swelling, and pain. In second degree burns, the epidermis and some portion of the underlying dermis are damaged, leading to blister and ulcer formation. Healing occurs in one to three weeks, usually leading to little or no scar formation.

In third degree burns, muscle tissue and bone may be involved, and secondary infection is common. It is in cases marked by significant tissue injury, and especially in cases involving infections, that topical oxygen/ozone/nitric oxide therapy finds the most usefulness. In the case of burns, the spectrum of pathogenic organisms may be wide and thus may be ideally suited for ozone therapy.

In burns, externally applied tri-gas concentrations need to be carefully calibrated. The clinician must be able to gauge the proper ozone concentration geared to the specific medical condition under treatment. In wet burns, for example, initial ozone concentrations will need to be low, in order to prevent inordinate systemic absorption through exudates absorption. As the burn heals and progressively dries, greater ozone concentrations may then be administered.

Nail Afflictions

Conditions implicating nails, which are therapeutically assisted by topical ozone treatment, include the following:

Candida albicans. Nails in this condition are painful, with swelling of the nail fold, and often, thickening and transverse grooving of the nail architecture. Loss of the nail itself may occur. Another frequent condition is Tinea Unguium, marked by thickened, hypertrophic, and dystrophic toenails. There are currently no topical antifungal agents of proven efficacy for this condition. Systemic anti-fungal agents show a spectrum of noxious side effects.

Tinea Pedis (Athlete's Foot). This very common disorder is caused by infection with species of Trichophyton, and with Epidermophyton floccosum. Chronic infection involving the webbing of the toes may evolve to secondary bacterial involvement. Lymphangitis and lymphadenitis may present themselves, as well as infection of the nails themselves (Tinea unguium; Onychomycosis). Nails may become thickened. yellow, and brittle. The patient may then develop allergic hypersensitivity to these organisms.

Topical oxygen/ozone/nitric oxide therapy offers unique treatment opportunities to these recalcitrant infections. Ozone penetrates the affected areas, including the nails proper, and with repeated administration. is capable of inactivating all species of fungi mentioned above.

Healing occurs slowly yet consistently, and skin integrity along with nail anatomy, gradually regain their normal configuration.

Radiodermatitis

This condition occurs during times when the body is exposed to ionizing radiation. This may result from radiological accidents or from radiation therapy. Radiation energy, imparted to cells, leads to cellular DNA injury.

Clinical findings are proportional to the type, amount, and duration of radiation exposure. Several clinical syndromes have been delineated, including radiation erythema, and radiodermatitis. While DNA damage cannot be easily repaired, secondary infections made more likely by decreased tissue resistance may be countered by topical ozone therapy. This avoids the systemic absorption of ointments and provides pan-pathogen protection.

Frostbite

Factors contributing to skin injuries due to cold derive from vasoconstriction and the formation of ice crystals within tissues. As frostbite progresses, loss of sensation occurs, and tissues become increasingly indurated to touch. Depending upon length of exposure, dry gangrene may develop. Dry gangrene may then evolve to wet gangrene if infection occurs.

Topical oxygen/ozone therapy has proven to be effective in decelerating or halting the pathogenesis of frostbite through (I) The immediate oxygenation of tissues, (2) Increasing blood flow through a direct vasodilatory effect upon the dermal arterioles, and (3) The prevention of secondary infection.

The present disclosure allows a microprocessor-controlled intra-envelope milieu geared to the therapy of frostbite, including proper temperature, humidity, and appropriate ozone/oxygen/nitric oxide concentrations.

Advantages of Oxygen/Ozone/Nitric Oxides Topical Wound Therapy

Topical oxygen/ozone/nitric oxide therapy (TGT) for the disorders mentioned above requires the precise diagnosis of the underlying conditions, and a correspondingly appropriately tailored treatment plan, which may include any one of several therapeutic modalities utilized concomitantly.

The salient advantages of tri-gas therapy (TGT) include:
The ease of administration of topical Tri-Gas Therapy.
TGT is an effective challenger to the viability of an enormous range of pathogenic organisms. In this regard, tri-gas therapy cannot be equaled. It is effective in a spectrum of aerobic and anaerobic bacterial organisms and a wide swath of viral families—lipid as well as non-lipid enveloped—as well as fungal families and protozoan pathogens. To duplicate this therapeutic action would require the administration of a large array of therapeutic agents belonging to antibacterial, antifungal, and antiparasitic groups.
Tri-gas therapy (TGT), appropriately applied in a timely fashion, may obviate the need for systemic antimicrobial therapy, thus saving the patient from the side effects this option could entail.
Tri-gas therapy (TGT) exerts its pa n-pathogenic actions through entirely different mechanisms than conventional antimicrobial agents. The latter must be constantly upgraded to surmount pathogen resistance and mutational defenses. TGT, on the other hand, presents direct oxidative challenge that cannot be circumvented by known mechanisms of pathogen resistance.
TGT makes use of a gas composition that, unlike many topical liquids—such as hydrogen peroxide—does not harm the integrity of healthy tissues.
Disadvantages of TGT include the fact that its gases have limited penetration into tissues, so that deeply ensconced microorganisms may escape its reach. Estimated penetration of the TGT gas mixtures into the dermis ranges from 2 mm to 0.5 cm. This feature, however, carries benefits in that the limited penetration of gases into the dermal skin layers signifies a minimal systemic penetration.

Nitric Oxide in Wound Healing

Nitric oxide is a vasodilator and an immune system modulator. Therapeutically, nitric oxide inhalation is currently used to treat hypoxic respiratory failure in the newborn. Applied topically to wounds, nitric oxide gas dilates superficial arterioles, increasing circulatory perfusion, thus exerting beneficial effects by bringing nutrients and immune factors to wounds, and by encouraging the clearing of wound debris and toxins.

Nitric oxide is a molecule with important biological functions. It is a cell signaling molecule, a neurotransmitter, an anti-oxidant, and an activator of immune system functions, notably involving interleukins and interferons.

Nitric oxide, because of its pulmonary vasodilating capacity, is currently used for treating respiratory distress syndrome (RDS) and persistent pulmonary hypertension (PPHN). In this proposed system, it can also be used to activate wound circulation and challenge the viability of microbes.

Applied topically as a gas to wounds, nitric oxide dilates arterioles, increasing the wound's circulatory inflow, and its vascular outflow. The wound thus benefits from the increased import of systemic oxygen and immune factors, and from the enhanced export of debris and toxins.

The system's incorporated oxygen concentrator removes most but not all nitrogen from the ambient air it processes. The small amount of residual nitrogen is converted to nitric oxide, as it is imparted electrical energy by the generator. Thus, the main therapeutic gases apposed to the wound include pure oxygen, ozone, and nitric oxide gas.

Tri Gas Therapy Dosages in Wound Healing

The intra-envelope gas mixture contains oxygen, ozone, nitric oxide, and nitrogen. In the presence of oxygen, some nitric oxide (NO) will convert to nitrogen dioxide (NO2).

The intra-envelope concentration of these gases is adjusted according to the wound's clinical status. As the wound's status changes during the course of therapy so may the configuration and concentrations of gases administered.

While oxygen is the most abundant gas in the treatment envelope, ozone concentrations may vary from 0% to 5% by volume. Nitric oxide concentrations may vary from zero to 1000 ppm. By comparison, pulmonary gas dosages in nitric oxide therapy are allowed to reach only 80 ppm for restricted amounts of time.

The central element of this invention rests on the use of selected topically applied gases that, alone and in combination, act to assist the wound healing process.

Encasing the wound under treatment, such as a diabetic or vascular skin ulcer, or an acute lesion due to combat injuries, is an envelope that holds the combination of gases so that they may be efficiently apposed to the lesion.

The principal active wound-healing gases within the envelope include oxygen (O2), ozone O3), and nitric oxide (NO).

Topical oxygen (O2) is already approved by the FDA for wound care, applied at atmospheric or at hyperbaric pressures. Oxygen serves to oxygenate tissues and, via its oxidant properties, exerts antimicrobial action. Oxygen, as such, can be present in the envelope, up to 100%.

Ozone (O3) is well known for its pan-antimicrobial action and for its capacity to enhance blood and tissue oxygenation. The concentration of ozone within the treatment envelope may be adjusted, reaching an upward limit of 5% by volume. In open wounds with active bleeding, ozone concentrations may be adjusted downward to 0.5% by volume to modulate absorption by blood. In such wounds, low ozone dosages may be used as prophylaxis against infections.

Chronic ulcers, on the other hand may need high initial ozone doses, reaching 5% by volume in order to penetrate biofilms and oxidize bacterial toxins.

Nitric oxide (NO) is a gas that is currently approved for the treatment of pulmonary hypertension in neonates and infants. As such, it is administered via the lungs in doses ranging from 5 ppm to 80 ppm. In these situations, vasodilation and bronchial relaxation produced by nitric oxide are life-saving.

In the healing of wounds, however, because skin tissues are much tougher than pulmonary epithelium, doses of NO gas may be much higher, ranging from a low 10 ppm to higher ranges of 10,000 ppm. A wound needing enhanced circulation may thus be eligible for more robust NO administration. Indeed, the activated circulation of wounds is therapeutic in that oxygen, nutrients and immune elements are dynamically delivered to the wound and toxins and waste products are efficiently eliminated.

These therapeutic gases may be delivered to the treatment envelope via dedicated oxygen and nitic oxide cylinders. The nitric oxide cylinder may be directly fed to the envelope. The oxygen source, however, will need to be delivered to a generator for conversion to ozone before entry into the envelope.

This invention proposes a novel formula for wound healing, namely by apposing a combination of oxygen, ozone and nitric oxide to wound tissues. The invention, however, also proposes a novel method for delivering these gases, namely an apparatus that can generate the therapeutic gas mixtures from ambient air rather than from dedicated gas supplies. The apparatus thus conceived, because it can be portable, allows it to be used in many situations, from clinics to battlefield theaters.

The following points describe and set forth certain elements of the invention but are not limiting of the scope of the invention.

1. The invention proposes an integrated self-contained unit that derives its final product, a mixture of wound healing gases, from ambient air. Via selective alteration of normal air's composition via the application of electrical energy, the gas components are modified so as to assume appropriate concentrations for wound healing. Ambient air is deliberately chosen as a source of gas because it contains essential gas precursor components for wound healing, namely oxygen and nitrogen.
2. This invention proposes a method of treating wounds with a mixture of (1) Oxygen (O2), (2) ozone (O3) and other oxygen reactive species, and, (3) nitric oxide (NO) and other nitrogen reactive species (NRS). In combination, the appropriate mixture of these gases, topically applied to skin wounds, embodies advantages that are not achieved by any of these individual gases used alone.
3. The three main gases generated for wound healing in this system are oxygen, ozone, and nitric oxide. This may be referred to as Tri-Gas Wound Therapy (TGWT). Other gases, in much more reduced concentrations, however. are also generated and possess biological effects. These include other members of Reactive Oxygen Species (ROS), and Nitrogen Reactive Species (NRS). In minuscule concentrations, the effects of these gases include antimicrobial actions and activation of healthy tissue physiology.
4. This therapy may be used to inactivate bacterial and fungal toxins. Secreted by many families of wound-invading bacteria and fungi, these toxins inhibit tissue healing via several mechanisms. Oxidation of toxins by ROS is the main mechanism for this action, while NRS species act synergistically.
5. This therapy may be used to dissolve bacterial biofilms. These protective films secreted by bacteria and fungi favor microbial growth by covering wounds with fibrin shields, thereby preventing wounds from exposure to oxygen healing action. The inclusion of NRS in the gas mixture, in combination with humidity, produces weak acids that dissolve biofilms.
6. This invention presents an innovative step for the following reasons: Whereas in previous systems the gas source was pure oxygen—usually provided by a medical grade oxygen cylinder—this system proposes the deliberate inclusion of small amounts of nitrogen for producing molecules in the nitrogen family capable of assisting in wound healing. Using ambient air as a gas feed not only makes the system portable and versatile, but also allows access to one of air's major component: Nitrogen.
7. The aim of this system is to provide wound care that addresses poorly healing wounds and chronically infected wounds such as decubitus ulcers. Representing major public health problems, these injuries, commonly derived from diabetes and vascular insufficiency, all too often result in dreaded limb amputations. This therapy aims to prevent limb amputations via accelerated wound closure.
8. Other indications for this system include the treatment of acute wounds, such as traumatic injuries and military combat/war wounds, and any wound that may require the prevention of infection.
9. Other pathologies amenable to this treatment system include but are not limited to: Burns, severe fungal infections, radiodermatitis, frostbite, gangrene and necrotizing fasciitis.
10. Tri-Gas Wound Therapy embodies several goals, namely: The increased oxygenation of injured tissues; the inactivation of wound pathogens; the enhancement of blood perfusion and circulation; the neutralization of bacterial and fungal toxins, and the dissolution of wound bacterial biofilms.
11. The ROS-NRS gas mixture can be mixed with water vapor for the creation of weak acids, such as nitric acid, that are applicable to wound healing.
12. A clear advantage of externally applied gas for wounds over liquid solutions is that the gaseous mixture interfaces with the wound evenly, reaching the wounds' interstices. One disadvantage is noted, namely that externally applied gases only penetrate 2 to 5 millimeters in depth. This is counterbalanced, however, by the advantage that the low depth of penetration reduces the possibility of systemic toxicity.
13. Tri-Gas wound therapy recruits the noted antimicrobial capacities of reactive oxygen species (ROS), in addition to those of nitrogen reactive species (NRS). Oxygen reactive species (ROS) are normally created in vivo by mammalian cell by enzymatic reactions, aiming to inactivate invading pathogens. Oxygen reactive species include, but are not limited to: Ozone, singlet oxygen, hydroxyl radical, peroxyl radical, peroxides and superoxide. While some of these compounds differ in their lifespan in the body and are usually evanescent, they all share a superior ability to extract electrons and thus offer potent antimicrobial oxidizing action. The Tri-Gas system is capable of selectively generating ROS and NRS and provide for their accurate dosing, appropriately dosed to the current clinical status of the wound.
14. Nitrogen reactive species (NRS) are nitrogen-containing molecules. Several possess anti-microbial actions. NRS are generated in vivo by mammalian organisms and serve a variety of functions, the most important of which are cell signaling, vasodilation and microbial inactivation. Increasing vascular perfusion through the presentation of nitric oxide allows for quicker wound resolution.
15. In order to generate these families of gases in concentrations appropriate for wound healing, the primary gas feed must contain both oxygen and nitrogen. This system thus starts its feed from ambient air that contains both. First filtered for purity by an air filter, ambient air is checked for humidity level via a sensor and adjusted accordingly. It is then fed to the system components for ROS and NRS generation.
16. The system comprises several components the first being an oxygen concentrator. This component removes nitrogen from ambient air, thereby providing the system with a gas mixture that contains mostly oxygen—up to 97%—with the rest nitrogen—up to 3%—and minuscule amounts of carbon dioxide.
17. The outflow from the oxygen concentrator is dehumidified for accurate ROS and NRS concentration accuracy, then delivered to the corona discharge unit that imparts it with electrical energy. Powered by a rechargeable battery or other source such as solar, the corona unit provides a high intensity electrical field through which the gas traverses. Adjusting amperage and voltage energy modulates the concentrations of generated ROS and NRS. Voltage regulation is integrated with gas flow data to create a gaseous mix optimal for the clinical situation at hand. Corona voltage is adjusted to yield desired concentration of oxygen, ozone, ROS. NO and NRS.
18. Aside from amperage and voltage, the generator may adjust for gas flow rate, for the nature of the dielectric, and for the configuration of the electrodes
19. Some corona discharge derivatives have little or no effect on wound dynamics and can be regulated by mitigation technologies, as is the case with nitrogen dioxide.
20. An air-cooling system is integrated to dissipate thermal excess.
21. The variable electrical energy from the corona unit transforms the configuration and the concentration of incoming gases. Oxygen, activated by corona discharge yields reactive oxygen species (ROS), while nitrogen yields nitrogen reactive species (NRS). A single source, ambient air, providing all the basic ingredients for this therapeutic wound healing mix, obviates the need for dedicated oxygen and nitrogen gas supplies. The oxygen concentrator may be adjusted to yield oxygen concentrations from 90% to 97%, or higher to the upper range of the concentrator's capacity. Correspondingly, the nitrogen concentration will vary inversely.
22. The gaseous mixture, as it exits the corona generator is adjusted for humidity. It may be quantitatively humidified to produce weak acids—such as nitric acid—which can be applied to the dissolution of wound bacterial biofilms.
23. The gas mixture is then dispatched to a treatment envelope encasing the wound under treatment. The envelope is chemically resistant to the tri-gas mixture. Duration of exposures to the gases is predicated on the wounds' clinical presentations and may be modified according to their evolution in time.
24. The apparatus also incorporates an oxygen analyzer, an ozone analyzer, and a nitric oxide analyzer. Thus, an accurate gauging of oxygen, ozone and nitric oxide gas concentrations can be ascertained, as they are applied to wounds in real time.
25. The system may be adjusted to address itself specifically to the therapy of circulatory disorders. In this clinical scenario, the system may be adjusted to deliver higher concentration of nitric oxide for longer periods of time, in order to enhance circulation and vascular perfusion.
26. The combination of ROS and NRS within the same envelope is likely to yield novel compounds that embody new antimicrobial properties.
27. The combination of ROS and NRS within the same envelope may yield novel compounds that embody tissue healing properties. properties
28. Oxygen itself is a recognized antimicrobial agent, especially effective when delivered to tissues under pressure, as in hyperbaric oxygen therapy. The proposed system delivers pure oxygen, along with other gases. Along with oxygen, ROS constitute an array of chemically active molecules that contain oxygen. Due to the their high oxidative properties, ROS chemically challenge the viability of a huge spectrum of microbes that commonly colonize wounds. ROS have the capacity to alter bacterial cell walls, bacterial cytoplasmic structures, bacterial genetic material. viral attachment proteins, viral transduction mechanisms, viral RNA and DNA, and fungal cell walls and organelles.
29. NRS, or nitrogen reactive species constitute an array of chemically active molecules that contain nitrogen. These include nitric oxide (NO) and nitrogen dioxide. NRS offer important advantages for the treatment of wounds. Recognized for their antimicrobial properties, they are also recruited herewith to enhance wound microcirculation. Nitric oxide dilates blood vessels, in turn encouraging the delivery of systemic oxygen and nutrients, and favoring the outflow of toxins and wound waste products.
30. The system is equipped with a heater/cooler unit for the gas outflow to wounds. The importance of this feature is that certain wounds—for example frostbite—may need graduated temperature adjustments during treatment. In addition, selected temperatures are optimal for maintaining the stability of gases. ROS, for example, are more stable at lower temperatures
31. The system is equipped with an outflow ozone neutralizer that has the capacity to deactivate ROS. Called “ozone destructors.” ROS deactivation units catalyze reactive oxygen species, thus accelerating their return to biatomic oxygen, and ensuring environmental safety.
32. The apparatus distinguishes itself by the fact that it provides for the analysis and automatic regulation of the gas milieu applied to wounds, throughout the duration of individual treatment, via ongoing feedback monitoring using sensors in the treatment envelope and servomechanisms.
33. The apparatus embodies a microprocessor with the capacity to evaluate incoming data from various gas analyzers, and to respond in a timely corrective fashion, in accordance with treatment protocols. Analyzers measure oxygen, ozone and nitric oxide levels. They may also monitor nitrogen dioxide.
34. The said microprocessor influences the functioning of the oxygen concentrator, the ozone/nitric oxide generator, the ozone destructor, the heater/cooler. and the humidifier. Ozone and oxygen concentrations, gas temperature, and gas humidity may thus be automatically modulated according to the changing conditions within the treatment envelope.
35. While oxygen is the most abundant gas in the envelope, ozone concentrations may vary from 0% to 5% by volume.
36. Nitric oxide concentrations may vary from zero to 1000 ppm. Nitric oxide is a vasodilator and an immunomodulator.
37. Applied topically to wounds, nitric oxide gas dilates superficial arterioles, increasing circulatory perfusion, thus exerting beneficial effects by bringing nutrients and immune factors to wounds, and by encouraging the clearing of wound debris and toxins. The apparatus herewith proposed generates ozone and nitric oxide gases, because the incorporated oxygen concentrator allows for some residual nitrogen to remain, allowing its conversion to nitric oxide (NO).
38. The microprocessor receives data from the bacterial gas sensor and, according to treatment protocol parameters, regulates the oxygen, ozone, and nitric oxide concentrations (via generator power modulation), and the length of treatment by modulating timer functions.

In one embodiment, the multi-gas mixture introduced into the treatment device for treatment of the wound comprises: oxygen gas by volume between 0% and 100% (e.g., 95% to 96%); ozone by volume between 0% and 5% (e.g., 4% to 5%) and nitric oxide by ppm between 0 ppm and 10,000 ppm (e.g., 0 to 1,000 ppm).

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not precludes the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. A system that is configured to generate a gaseous mixture from ambient air for treatment of a wound comprising:
a housing that includes an inlet for receiving ambient air that includes oxygen gas and nitrogen gas, the inlet being in communication with a first conduit;
an oxygen concentrator receives the ambient air from the first conduit and is configured to concentrate an oxygen concentration of the ambient air and generate a first output gas;
a corona discharge generator that is fluidly connected to an outlet of the oxygen generator by a second conduit, the corona discharge generator being configured to generate a second output gas from the first output gas by converting portions of the concentrated oxygen into reactive oxygen species (ROS), and converting portions of the nitrogen gas into nitrogen reactive species (NRS);
a treatment device for receiving the wound to be treated, the treatment device being in fluid communication with an outlet of the corona discharge generator and receives the second output gas including the ROS and NRS within a multi-gas compartment within a housing of the treatment device; and
an ozone destructor that is in fluid communication with a fourth conduit with is coupled to an outlet of the treatment device, the ozone destructor being configured to convert ozone that forms part of the ROS to pure oxygen, releasing it back to ambient air.

2. The system of claim 1, further including:
an air filter located within the first conduit; and
a fan and flow meter located along the first conduit downstream of the air filter, the fan and flow meter regulating an inflow of the ambient air and delivers the ambient air to the oxygen concentrator.

3. The system of claim 1, further including:
a first humidifier/dehumidifier unit that is located within the second conduit for adjusting a humidity level of the first output gas; and
a second humidifier/dehumidifier unit that is located within the third conduit for adjusting a humidity level of the second output gas.

4. The system of claim 1, further including a temperature controller or regulator that is disposed along the third conduit for heating or cooling the second output gas prior to delivery to the treatment device.

5. The system of claim 4, wherein the temperature controller or regulator comprises a heat exchanger.

6. The system of claim 1, further including a plurality of sensors located within the multi-gas compartment for monitoring properties of the second output gas within the multi-gas compartment.

7. The system of claim 6, wherein the plurality of sensors includes gas sensors including at least one of an oxygen sensor, an ozone sensor, and a nitric oxide to measure oxygen gas, ozone gas, and nitric oxide gas in the second output gas that is delivered into the multi-gas compartment.

8. The system of claim 1, wherein the corona discharge generator is powered by a power source that comprises one of a battery and dedicated power source in a room.

9. The system of claim 1, wherein the oxygen concentration of the first output gas is between 90% to 97% by volume.

10. The system of claim 1, wherein a nitrogen concentration of the first output gas is up to 4% by volume.

11. The system of claim 1, further including a main controller that monitors one or more properties of the second output gas within the multi-gas compartment.

12. The system of claim 11, wherein the main controller includes a display for displaying values of the one or more properties.

13. The system of claim 12, wherein the one or more properties of the second output gas includes an oxygen concentration, an ozone concentration, and a nitric oxide concentration of the second output gas that is delivered into the multi-gas compartment.

14. The system of claim 13, the ozone concentration of the second output gas is between 0% to 5% by volume.

15. The system of claim 13, wherein the nitric oxide concentration is between 0 to 1000 ppm.

16. The system of claim 1, wherein the second output gas that is delivered to the treatment device includes oxygen gas, ozone gas and nitric oxide gas.

17. A method for treating a wound comprising the steps of:
introducing ambient air into an oxygen concentrator that increases an oxygen concentration of the ambient air and forms a first output gas;
introducing the first output gas into a corona discharge generator that transforms the first output gas into a second output gas by converting portions of the concentrated oxygen into reactive oxygen species (ROS), and converting portions of the nitrogen gas into nitrogen reactive species (NRS); and
delivering the second output gas into a multi-gas compartment within a treatment device in which the wound is positioned.

18. The method of claim 17, wherein the ROS includes ozone and the NRS includes nitric acid.

19. The method of claim 17, further including the step of:
converting at least a portion of ozone that is removed from the multi-gas compartment into pure oxygen gas by an ozone destructor that is in fluid communication with an outlet of the treatment device.

20. The method of claim 17, further including the steps of:
altering a humidity level of the first output gas using a first humidifier/dehumidifier that is located between the oxygen concentrator and the corona discharge generator; and
altering a humidity level of the second output gas using a second humidifier/dehumidifier that is located between the corona discharge generator and the treatment device.

21. The method of claim 17, further including the step of:
regulating a temperature of the second output gas with a temperature regulator prior to introduction into the treatment device.

22. The method of claim 17, further including the step of:
monitoring one or more properties of the second output gas within the multi-gas compartment using a plurality of sensors that are in communication with a main controller.

23. The method of claim 22, wherein the one or more properties of the second output gas includes an oxygen concentration, an ozone concentration, and a nitric oxide concentration of the second output gas that is delivered into the multi-gas compartment.
